(19) United States
(12) Patent Application Publication
(10) Pub. No.: US 2017/0247648 A1
(43) Pub. Date: Aug. 31, 2017

(54) METHOD AND ALGAL GROWTH SYSTEM FOR AUTOTROPHIC ALGAL GROWTH

(71) Applicant: PIARCS, PBC, Boulder, CO (US)

(72) Inventor: Alexandra D. Holland, Boulder, CO (US)

(21) Appl. No.: 15/517,105

(22) PCT Filed: Oct. 14, 2014
(86) PCT No.: PCT/US2014/060491

§ 371 Date: Apr. 5, 2017

(51) Int. Cl. C12M 1/00; C12N 13/00; C12M 1/34

(52) U.S. Cl. CPC C12M 21/02; C12M 29/06; C12M 31/02; C12M 47/10; C12M 41/34; C12N 13/00; C12M 41/06; C12M 23/34; C12M 23/22

(57) ABSTRACT

Autotrophic algal growth in high incident light situations may be conducted in a reactor with circulation of algal reaction medium between light and dark zones with very short residence time in the light zone to maintain algal growth in the reactor in a linear growth regime in which the rate of algal biomass production is proportional to the incident photosynthetic photon flux density. Process monitoring and control may permit quick processing in a single step even in open pond systems. Dissolved nitrogen levels in product may be monitored and nitrogen nutrient input may be restricted to reduce dissolved nitrogen in effluent and to increase lipid yield without a separate nitrogen starvation step.

### FIELD OF THE INVENTION

The invention relates to methods and systems for autotrophic algal growth, including for use in open pond systems.

### BACKGROUND

The prospect of autotrophic (or light-driven) algal biomass production as a sustainable substitute for fossil feedstocks holds promise, but has yet to fulfill its potential. Inputs for autotrophic algal growth include photosynthetically active radiation (PAR) that is absorbed by the algae as energy for growth and chemical nutrients, including nitrogen, phosphorous and other nutrients. Generally, photosynthetically active radiation is electromagnetic radiation within a wavelength range of about 400 nm to about 700 nm. For purposes of biofuel production, it is generally desirable that the algae contain a high lipid (oil) fraction, but obtaining a larger lipid fraction in algae product is often achieved at the expense of total biomass yield. Development of autotrophic algal growth systems that efficiently use inputs to generate a high biomass yield and with a high lipid content has been a challenge. At present, there are two basic reactor system approaches to algal growth. One approach is to use open pond systems with natural sunlight as the sole or primary light source. So-called “raceway” pond systems are typical of this approach. A second approach is to use a closed bioreactor system, in which the reactor is not open to the outside environment and all conditions are carefully controlled. Much research has gone into developing a number of different closed bioreactor systems.

Open pond systems have advantages of low cost (both for capital and operating expenditures) and general simplicity of design and operation. Open pond systems, however, have significant disadvantages in terms of process control, algae biomass yield and space requirements. Such open systems depend upon natural sunlight as a source of PAR, which is inherently variable due to weather and seasonal changes, and the systems are unproductive during nighttime hours. Pond systems are planar reactor systems and receive the full intensity of natural sunlight through a planar liquid surface at the top of the pond. A parameter that may be used to quantify PAR received through such a planar surface is the photosynthesis photon flux density (PPFD), expressed in microeinsteins per square meter per second (μE m⁻² s⁻¹). An Einstein is a unit of light equal to one mole of PAR photons. During times of high solar insolation, the incident PPFD on the algal medium may be in a light excess condition that supports exponential algal growth in a light zone at the top of the pond where algae concentration is a limiting factor to ability of the algae to use all of the incident light, whereas at times of low solar insolation, a light limitation condition may exist in which the lower level of incident PPFD may not be sufficient to support such exponential growth and algal growth may proceed in a linear manner proportional to the intensity of the incident PPFD received at the surface of the pond. At times of very high solar insolation, the level of incident PPFD may be at a level that is inhibitory to algal growth, resulting in a potentially significant drop in algae biomass yield per photon of PAR. Additional information on the effect of such light excess and light limitation conditions is provided in Holland A D, Wheeler D R (2011) Intrinsic autotrophic biomass yield and productivity in algae: Modeling spectral and mixing-rate dependence. Biotechnol J 6:584-599; and in Holland A D, Dragavon J M, Sigee D C (2011) Intrinsic autotrophic biomass yield and productivity in algae: Experimental methods for strain selection. Biotechnol J 6:572-583. Efficient use of nutrients in open systems may also be difficult to achieve due to the variable nature of light and variability of other ambient conditions (e.g., temperature) in an open system. In addition to the cost of nutrients not consumed in algal growth processing, inefficient use of nutrients can also lead to environmental concerns with nutrient levels in water effluent from such systems. Open systems are also not suitable for use in all geographic locations. Moreover, a significant problem with open systems is invasion by unwanted microbes that compete with desired algae strains for available light and nutrients, as well as invasion by grazers that feed on the algae, both of which can significantly reduce algal biomass yield. Competition by cyanobacteria, for example, is often a significant problem.

Because of the numerous technical disadvantages associated with open pond systems, there is significant interest in alternative systems in the form of closed bioreactor systems. Closed bioreactor systems address many of the technical disadvantages through precise control of operating conditions, which can lead to higher algae biomass yield. Such closed bioreactor systems typically use an artificial light source, either as a supplement for natural sunlight or as a sole light source and may avoid processing complications associated with the variable light situation in open systems. Nutrient feed levels may be more closely matched to algal growth needs under the controlled conditions of the closed system, leading to a more efficient use of nutrients. Closed systems also permit significant reduction or elimination of problems associated with invasion of unwanted microbes and better control on light insolation conditions for more precise control of algae growth conditions. The main disadvantage of closed bioreactor systems is high cost. Both capital and operating expenditures tend to be significantly higher than for open pond systems. Moreover, although the use of artificial light permits precise control of light delivery for algal growth, the use of artificial light sources is expensive, both in terms of lighting hardware and electricity charges for generating artificial light.

There is a significant need for improved reactor system designs and processes that may be applied to open pond systems under conditions of natural sunlight to better take advantage of the cost advantages of such systems while addressing technical operating disadvantages of such systems compared to closed bioreactor systems.

### SUMMARY OF THE INVENTION

Autotrophic algal growth processing may be enhanced, including in open pond reactor systems, through controlled circulation, or cycling, of algae-containing reaction medium between a light reactor zone and a dark rector zone to take advantage of algal growth potential of high incident PPFD levels available from natural solar radiation. Mixing rates between the light and dark zones in particular may have a significant effect on efficient utilization of incident PAR in such high incident PPFD situations. Through appropriate control of mixing conditions, even under high incident PPFD situations, algal growth in a reactor may be maintained in a linear growth regime, in which the rate of algal biomass production is proportional to the incident PPFD. Reference is made to Algal Biorefineries, Volume I: Cultivation of Cells and Products; Bajpai R, Prokop A, Zappi, M. (Eds.); Oct. 15, 2013; chapter titled “Algal Reactor Design Based on Comprehensive Modeling of Light and Mixing” by Holland A D and Dragavon J M, pp 25-68; the entire contents of which are incorporated herein by reference for all purposes.

Disclosed herein are methods for autotrophic algal growth and algal growth systems useful or autotrophic algal growth processing.

A first aspect of this disclosure is provided by various methods for algal growth, in which each of the various methods comprise:
circulating an algae-containing reaction medium between a light reactor zone and a dark reactor zone of a reactor volume of an algal growth reactor;
during the circulating, adding to the reaction medium nutrients for algal growth in the reaction medium, the nutrient comprising at least a nitrogen nutrient; and
during the circulating, irradiating the reaction medium in the light zone of the reactor with photosynthetically active radiation for absorption by algae in the algae-containing medium for algal photosynthesis.

A number of feature refinements and additional features are applicable to the methods of the first aspect. These feature refinements and additional features may be used individually or in any combination. As such, each of the following features may be, but are not required to be, used with any other feature or combination of any method of the first aspect or the subject matter of any other aspect of the disclosure.

In some preferred implementations, a method may include, during the circulating, operating the reactor under a linear growth regime, in which the rate of algal biomass production is proportional to incident PPFD on the reaction medium. Promoting a linear growth regime even during times of high incident PPFD may include maintaining a very short residence time of reaction medium in the light zone.

A method may include, during the circulating, maintaining a first residence time of the reaction medium in the dark reactor zone of at least 0.2 second and a second residence time of the reaction medium in the light zone of not more than 5 milliseconds. Such a first residence time may be at least 0.2 second, at least 0.5 second, at least 1 second, at least 2 seconds or at least 3 seconds. Such a first residence time may often be not more than 5 seconds, not more than 4 seconds or not more than 3 seconds. Such a second residence time may be not more than 5 milliseconds, not more than 4 milliseconds, not more that 3 milliseconds, not more than 3 milliseconds or not more than 1 millisecond. Such a second residence time may often be at least 0.02 milliseconds, at least 0.1 millisecond, at least 0.5 millisecond, at least 1 millisecond or at least 2 milliseconds. The first residence time and the second residence time may be such that a ratio of the first residence time to the second residence time may be at least at least at least 100:1, at least 1,000:1, or more.

The circulating may include sparging gas into the reaction medium at a gas velocity of at least 2 meters per second, at least 5 meters per second, at least 10 meters per second, at least 20 meters per second at least 40 meters per second or at least 80 meters per second. Such a gas velocity may be not more than 200 meters per second, not more than 100 meters per second, not more than 50 meters per second or not more than 25 meters per second or not more than 15 meters per second. Reference to the gas velocity of the sparge gas refers to the velocity of the gas as it exits from a gas delivery port, also referred to herein as an orifice. The sparging may include introducing the sparge gas into the reaction medium from gas delivery ports having a maximum cross-dimension perpendicular to a direction of flow (e.g., diameter of circular orifice, diagonal of a square orifice) in a range having a lower limit of 1 micron, 2 microns, 5 microns, 10 microns, 20 microns, 50 microns or 100 microns and an upper limit of 200 microns, 100 microns, 75 microns, 50 microns, 25 microns and 15 microns; provided that the upper limit is larger than the lower limit. Sparge gas delivery ports may be provided in an array at density of ports per square meter in a range having a lower limit of 200, 500, 1,000, 5,000 or 10,000 and un upper limit of 20,000, 10,000, 5,000, 2,000 or 1,000; provided that the upper limit is larger than the lower limit. The gas delivery ports may be provided in arrays of varying configurations. Spacing between ports may be uniform or varying. Ports may be provided in spaced rows of ports (e.g., rows of orifices on a gas delivery conduit), with a spacing between rows being uniform or varying and with a spacing of ports within a row being uniform or varying. A spacing between ports in a row may be smaller than the spacing between rows. A spacing between rows may be at least 1.5 times as large as the spacing between ports in a row. By spacing of ports (orifices), or rows of ports, it is meant center-to-center spacing, unless otherwise specifically indicated in the circumstance.

Sparge gas introduced to drive circulation between a light reactor zone and a dark reactor zone may be introduced into or slightly below the light reactor zone. In some preferred implementations, such sparge gas is introduced into the reaction medium just below the bottom of a light zone of the reaction medium. The sparge gas may be introduced into the reaction medium at a quiescent depth in the reaction medium that is not larger than 10 centimeters, not larger than 8 centimeters, not larger than 6 centimeters or not larger than 4 centimeters, although such a quiescent depth may be at least 1 centimeter, at least 2 centimeters or at least 3 centimeters. By quiescent depth, it is meant the depth in the reaction medium assuming the reaction medium is in a quiescent state in the reactor with the sparge gas turned off and with the reaction medium not otherwise being agitated in the reactor. As will be appreciated, during algal growth operations with the sparger turned on, the reaction medium will not be in a quiescent state, but specifying a depth relative to the quiescent state is useful for design reference purposes.

Sparge gas introduced to drive circulation between a light reactor zone and a dark reactor zone may be any gas composition, and preferably may include some carbon dioxide for use in algal growth. Such a sparge gas containing carbon dioxide may conveniently be air, which contains a small quantity of carbon dioxide, or may be a gas with a higher carbon dioxide level.

In addition to gas sparging to drive circulation between light and dark reactor zones (which may for convenience be referred to as first sparging with a first sparge gas), the method may also include a second sparging of a second sparge gas, which may be the same or different composition than the first sparge gas. The second sparging may be at a lower elevation in the algal growth reactor than the first sparging. The second sparging may assist circulation of reaction medium through the dark reactor zone and back to a vicinity of the first gas sparging for further circulation through the light zone. The second sparge gas may also provide a primary source of carbon dioxide for use in algal growth, and the second sparge gas may have a higher carbon dioxide content than the first sparge gas. The second sparge gas may have a carbon dioxide concentration of at least 0.4 volume percent, at least 1 volume percent, at least 10 volume percent or at least 25 percent, or more. The velocity of the first sparge gas to drive circulation between the light reactor zone and the dark reactor zone will typically be much higher than the velocity of such a second sparge gas. A ratio of the first gas velocity to the second gas velocity may be at least 5:1, at least 10:1, at least 25:1, at least 100:1, or more.

Instead of, or in addition to, use of a second sparge gas to assist circulation of reaction medium through the dark reactor zone, other mechanical mixing techniques may also be employed, such as mixing impellers or circulation pumps.

Irradiating the reaction medium in the light reactor zone with photosynthetically active radiation may include a high incident PPFD for absorption by algae in the reaction medium for algal photosynthesis, such as may occur during times of high solar insolation. Such an incident PPFD may be at least 500, at least 1000, at least 1500 or even at least 2000 microeinsteins per square meter per second (μE m⁻² s⁻¹). In some instances incident PPFD from natural solar radiation may be as high as around 2500 μE m⁻² s⁻¹. Even under such conditions of high incident PPFD, a method may include, during the irradiating, maintaining a residence time in the light reactor zone to maintain a linear growth regime in the reactor where the rate of algal biomass production is proportional to the incident PPFD. The irradiating may be conducted continuously for at least four daylight hours per day for multiple consecutive days at an incident PPFD from natural sunlight of greater than 500, greater than 750, greater than 1,000 or even greater than 1,500 μE m⁻² s⁻¹.

A method may include, during the irradiating, fluorometrically monitoring the reaction medium and adjusting at least one operating parameter of the reactor in response to a change in a monitored fluorometric property of the reaction medium. Fluorometric monitoring may be or include fluorometric monitoring the reaction medium in the light reactor zone. Fluorometric monitoring may include subjecting a slipstream of reaction medium from the light reactor zone to excitation radiation and detecting fluorescent response to the excitation radiation, for example by pulse-amplitude modulated fluorometry. The fluorometric monitoring may include passive monitoring, for example monitoring fluorescence of the reaction medium in the light reactor zone due to the photosynthetically active radiation (e.g., natural sunlight) incident on the reaction medium. A control adjustment based on changes in a monitored fluorometric property may include changing residence time of reaction medium in the light reactor zone. A higher monitored fluorescent emission from the light reactor zone may indicate loss of incident PAR due to non-photochemical quenching (i.e., heat), and the adjustment may include decreasing the residence time of the reaction medium in the light reactor zone in response to an increase in monitored fluorescence of the reaction medium during the fluorometric monitoring.

A method may include introducing reaction medium from the dark reactor zone into the light reactor zone at a velocity of the reaction medium into the light reactor zone at a high velocity, for example at a velocity of at least 1 meter per second, at least 5 meters per second or at least 10 meters per second. Such a velocity may often be not larger than 30 meters per second or not larger than 20 meters per second.

A method may include, during the circulating, a ratio of a first volume of reaction medium contained in the dark reactor zone and a second volume of the reaction medium contained in the light reactor zone may be at a ratio of at least 5:1, at least 10:1 or at least 25:1. Such a ratio may often be not larger than 100:1.

The algal growth reactor may include a reactor vessel in which the light reactor zone is disposed at a higher elevation within the reactor vessel than the dark reactor zone within the reactor vessel, and the irradiating may be or include receiving natural sunlight into the reactor vessel from above. PAR received by the reaction medium in the reactor vessel may or may not include artificial light from an artificial light source, instead of or in addition to natural sunlight. However, in preferred implementations the PAR received by the reaction medium includes natural sunlight or includes only natural sunlight. Such a reactor vessel may be optically open to receive the natural sunlight only from above. The reactor vessel may be covered from above to prevent dilution of reaction medium by rainwater and/or to increase humidity above the reaction medium to reduce evaporative losses, provided that such a cover provides an optically transmissive path for sunlight to pass through the cover to reach the reaction medium. Such a reactor vessel may be or include a pond, which may be open, covered or partially covered. A pond may have suitable fluid-containment walls, for example cement or concrete walls or a plastic liner.

A method may include, during the circulating, removing a portion of the reaction medium from the algal growth reactor as reactor product. The reactor product may be used as or processed to prepare further products. Algae in the reactor product may be lysed and the lysed material subjected to further processing to recover a lipid fraction from the lysed algae, as may be desirable for use as or for further processing to prepare a biofuel.

A method may include monitoring a dissolved nitrogen level in the reaction medium, either in the reactor or outside of the reactor, and adjusting an amount of the nitrogen nutrient added to the reaction medium during the adding to maintain the dissolved nitrogen at a desired level, for example in a desired predetermined range in a reactor product. Such monitoring could involve monitoring a concentration of nitrogen in liquid of the reactor product. The dissolved nitrogen concentration may be maintained in the reactor product at a concentration of no larger than 1 milligram, no larger than 800 micrograms, no larger than 700 micrograms, no larger than 600 micrograms or no larger than 500 micrograms of dissolved nitrogen per liter of the liquid. During the circulating, the reactor may be operated at a nitrogen quotient in a range of from 50% to 95% of a nitrogen quotient for the same algal culture of the reaction medium processed in the reactor under nitrogen excess and reactor operating conditions otherwise the same, wherein the nitrogen quotient is in grams of nitrogen in the biomass of the reactor product per gram of the biomass on a dry weight basis. Operating the reactor with at a slightly limited nitrogen level relative to a nitrogen replete level may provide reactor product with a higher lipid content without the extra step of nitrogen starvation as with prior art processes. Such operation at a low nitrogen quotient to produce a reactor product with high lipid content benefits from operation of the reactor in a linear growth regime in which the rate of algal biomass production is proportional to the incident PPFD

An advantage of operating with a high shear environment in the light zone such as may occur through introduction of high velocity sparge gas to promote high liquid velocities through the light reactor zone is that eukaryotic algae may be grown under significantly reduced problems with contaminating microbes such as cyanobacteria, even in a reactor volume that is open to the exterior environment, such as open pond configurations. Such a high shear zone may not be problematic for eukaryotic algae, but is detrimental to cyanobacteria and may significantly suppress cyanobacteria growth in competition with the desired eukaryotic algae. Even in an open system, at least 90 weight percent of biomass, on a dry weight basis, in recovered reactor product may be eukaryotic algae.

The reaction medium may be an algal culture including any desired algae. The reaction medium may include any biomass concentration. A typical range of biomass concentrations is from 2 to 10 grams of biomass (on a dry weight basis) per liter of the reaction medium.

The light reactor zone may typically have a much smaller depth below a level of incident PAR than a depth of the dark reactor zone below the light reactor zone. As used herein, a light reactor zone, or simply light zone, is a zone within the reactor occupied by reaction medium in which PPFD in the reaction medium is at least 50 μE m⁻² s⁻¹ and a dark reactor zone, or simply dark zone, is a zone within the reactor occupied by reaction medium in which PPFD in the reaction medium is smaller than 50 μE m⁻² s⁻¹. The reaction medium in the light reactor zone in a method may have a quiescent depth of not larger than 8 centimeters, not larger than 6 centimeters or not larger than 4 centimeters. During nighttime hours a reaction medium may have no light zone when the sole light source for the reactor is natural solar radiation. In contrast, even during daylight hours with high incident PPFD, a dark reactor zone may often have a depth from top to bottom in a range of from 20 centimeters to 100 centimeters.

A method may include monitoring one or more property during autotrophic algal growth processing, for example a fluorometric property as discussed above, and adjusting one or more operating parameter based on changes in a monitored property or properties. A method may include monitoring incident PPFD to the reaction medium and adjusting at least one operating parameter of the reactor based on changes in the monitored incident PPFD, including at least one operating parameter selected from the group consisting of residence time of the reaction medium in the light reactor zone, rate of addition of nitrogen nutrient, depth of liquid in the light reactor zone and combinations thereof. A method may include increasing a rate of addition of nitrogen nutrient (and/or another nutrient) in response to a monitored increase in the incident PPFD and decreasing the rate of addition of the nitrogen nutrient (and/or another nutrient) in response to a monitored decrease in the incident PPFD. A method may include decreasing residence time of the reaction medium in the light reactor zone in response to a monitored increase in the incident PPFD and increasing the residence time of the reaction medium in the light reactor zone in response to a monitored decrease in the incident PPFD.

A second aspect of this disclosure is provided by various algal growth systems for autotrophic growth, wherein each of the various systems comprise:
an algal growth reactor with an internal reaction volume to receive and contain algae-containing reaction medium during autotrophic algal growth;
the reactor comprising a first reactor portion including a first portion of the internal reaction volume to provide a dark reactor zone for the reaction medium during autotrophic algal growth;
the reactor comprising a second reactor portion including a second portion of the internal reaction volume to provide a light reactor zone for the reaction medium during autotrophic algal growth;
a light transmissive path in optical communication with the second portion of the internal reaction volume to provide photosynthetically active radiation from a light source to the light reactor zone of the second portion of the internal reaction volume to be absorbed by biomass in the second portion of the internal reaction volume during autotrophic algal growth; and
a liquid circulation system to circulate the reaction medium during autotrophic algal growth between the dark reactor zone in the first portion of the internal reaction volume and the light reactor zone in the second portion of the internal reaction volume.

A number of feature refinements and additional features are applicable to the algal growth systems of the second aspect. These feature refinements and additional features may be used individually or in any combination. As such, each of the following features may be, but are not required to be, used with any other feature or combination of any algal growth system of the second aspect or with subject matter of any other aspect of the disclosure.

The reactor may include any features or features, in any combination, of a reactor as described with respect to the first aspect, including but not limited to the internal reaction volume, reaction medium, light reactor zone, dark reactor zone and liquid circulation (including with respect to gas sparging).

The liquid circulation system may include a gas sparge system to sparge pressurized gas into the internal reaction volume between the first portion and the second portion of the internal reaction volume to drive circulation of the reaction medium between the dark zone in the first portion of the internal reaction volume and the light zone in the second portion of the internal reaction volume during autotrophic algal growth. The gas sparge system may have any feature or features or perform in any manner as discussed in relation to the first aspect. The gas sparge system may be a first gas sparge system and the pressurized gas may be a first pressurized gas, and the algal growth system may include a second gas sparge system to sparge a second pressurized gas into the dark zone of the first portion of the internal reaction volume during autotrophic growth in the internal reaction volume. Such a second gas sparge system may have any feature or features or perform in any manner as discussed with respect to the first aspect in relation to second gas sparging.

The liquid circulation system may circulate the reaction medium during autotrophic algal growth between the dark zone of the first portion of the internal reaction volume and the light zone of the second portion of the internal reaction volume at a residence time in the second portion of the internal reaction volume of no more than 5 milliseconds and a residence time in the first portion of the internal reaction volume of at least 0.2 second, or any such other residence times as discussed in relation to the first aspect.

An algal growth system may include:
a monitoring system to monitor one or more property of performance of the reactor during autotrophic algal growth in the internal reaction volume and to generate and transmit electronic data signals indicative of the one or more monitored property; and
a computer controller system in electronic communication with the monitoring system to receive the electronic data signals and to generate electronic control signals to adjust one or more reactor operating parameters to control autotrophic algal growth in the internal reaction volume.

The monitoring system may include any feature or features or may operate in any manner as discussed in relation to the first aspect.

The computer controller system may include a computer processor and non-volatile computer memory with instructions executable by the computer processor to evaluate the electronic data signals and generate the electronic control signals. Such instructions may include instructions for evaluating the electronic data signals and generating the electronic control signals to maintain operation of the reactor under a linear growth regime where the rate of algal biomass production is proportional to the incident PPFD.

The monitoring system may include an incident light monitoring unit to monitor incident PPFD received by the light zone during autotrophic algal growth in the internal reaction volume; and the computer controller system may be in electronic communication with the incident light monitoring unit and the electronic data signals include electronic signals from the incident light monitoring unit indicative of the monitored PPFD. The computer controller system may be in electronic communication with the monitoring unit to receive the electronic signals indicative of the monitored PPFD and to generate electronic control signals to adjust a rate of addition of nitrogen nutrient in response to a monitored change in the incident PPFD. The computer controller system may be in electronic communication with the incident light monitoring unit to receive the electronic signals indicative of the monitored incident PPFD and to generate electronic control signals to adjust the residence time of the reaction medium in the light zone based at least in part on the monitored incident PPFD.

The monitoring system may include a dissolved nitrogen monitoring unit to monitor a concentration of dissolved nitrogen in liquid of the reaction medium; and the computer controller system may be in electronic communication with the dissolved nitrogen monitoring unit, the electronic data signals may include electronic signals from the dissolved nitrogen monitoring unit indicative of the monitored dissolved nitrogen concentration and the electronic control signals may include electronic signals directed to maintaining the dissolved nitrogen concentration at a concentration within a desired range of dissolved nitrogen per liter of the liquid in the reaction medium (e.g., a dissolved nitrogen concentration of no larger than 700 micrograms of dissolved nitrogen per liter of the liquid). An algal growth system may include a nutrient supply system in fluid communication with the interior reaction volume to supply nutrients including at least nitrogen nutrient during autotrophic algal growth in the internal reaction volume; and the computer controller system may be in electronic communication with the nutrient supply system to provide electronic control signals to the nutrient supply system to adjust a level of nitrogen nutrient supplied to the internal reaction volume based at least in part on the monitored dissolved nitrogen concentration.

The monitoring system may include a fluorometric monitoring unit to monitor at least one fluorometric property of the reaction medium in the reactor volume and the electronic data signals include electronic signals indicative of the monitored at least one fluorometric property; and the computer controller system may be in electronic communication with the fluorometric monitoring unit to receive the electronic signals indicative of at least one monitored fluorometric property and to generate electronic control signals to adjust the residence time of the reaction medium in the light zone based at least in part on the monitored at least one fluorometric property. The fluorometric monitoring unit may include a pulse-amplitude modulated fluorometer and the electronic data signals include electronic signals indicative of monitored pulse-amplitude modulated fluorometric data from the pulse-amplitude modulated fluorometer. The fluorometric monitoring unit may be fluidly connected with the second portion of the internal reaction volume to sample reaction medium in the light zone for fluorometric monitoring. The fluorometric monitoring unit may include a fluorometer disposed to monitor fluorescent emission from reaction medium in the light zone due to excitation by photosynthetically active radiation incident upon the reaction medium from the light transmissive path.

The depth (vertical thickness) of the light reactor zone may be significantly smaller than the depth (vertical thickness) of the dark reactor zone, as discussed above with the methods of the first aspect. The internal reaction volume may include a ratio of the volume of the first portion of the internal reaction volume to the volume of the second portion of the internal reaction volume of at least 5:1. An algal growth reactor may have a reactor vessel in which the second portion of the internal reaction volume is disposed at a higher elevation within the reactor vessel than the first portion of the internal reaction volume; and the light transmissive path may be optically open to receive natural sunlight from above to irradiate the light zone during daylight hours (e.g., a pond system using natural sunlight). The internal reaction volume may contain reaction medium including algae dispersed in aqueous liquid. The reactor may be a planar reactor (e.g., a pond). An algal growth system may include a product recovery system in fluid communication with the internal reaction volume to receive at least a portion of reaction medium as reactor product and to lyse algae in the reactor product and prepare a lipid fraction from the lysed algae.

References to electronic communication and electronic signals refer also to alternative implementations in which the communication may be optical communication and the signals may be optical signals.

These and other aspects, and additional features of such aspects, are further described below with reference to the drawings and in the claims.

### BRIEF DESCRIPTION OF THE DRAWINGS

FIG. 1 illustrates an embodiment of an algal growth reactor and algal growth processing using the algal growth reactor.

FIGS. 2-4 illustrates another embodiment of an algal growth reactor and algal growth processing using the algal growth reactor.

FIG. 5 illustrates some details of a gas sparge system of the algal growth reactor and algal growth processing of FIGS. 2-4.

FIGS. 6-8 illustrate various embodiments for configurations for gas delivery orifices for gas sparging into reaction medium to drive reaction medium circulation between light and dark zones of an algal growth reactor.

FIG. 9 illustrates an example embodiment of an algal growth system and algal growth processing using the algal growth system.

### DETAILED DESCRIPTION

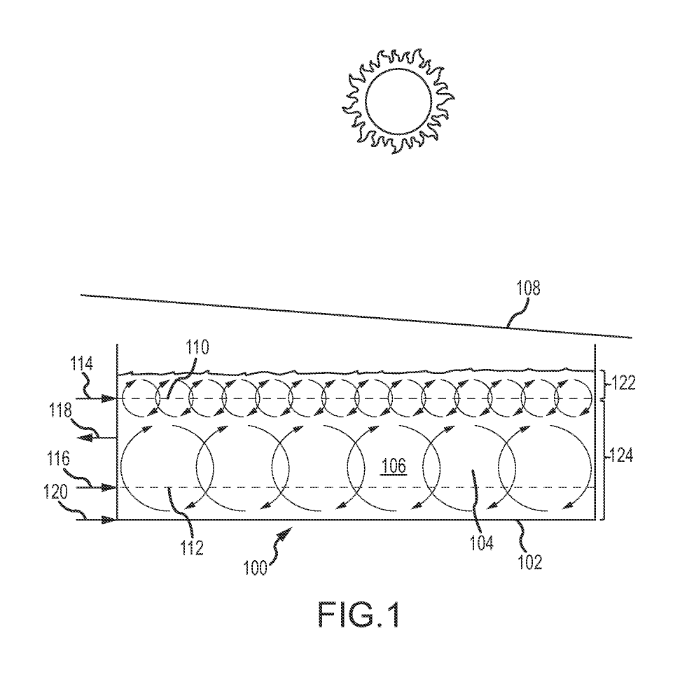

FIG. 1 generally illustrates an example embodiment of an algal growth reactor 100 that includes a liquid-containment vessel 102 having an internal reaction volume 104 in which is contained a reaction medium 106 including algae disbursed in aqueous liquid. The top of the vessel 102 is covered by a cover 108 that prevents rain from accumulating in the internal reaction volume 104 and diluting the reaction medium 106 and increases humidity above the top of the reaction medium 106 to reduce evaporation of aqueous liquid from the reaction medium 106. The cover 108 is optically transmissive (transparent) to permit solar radiation to pass through the cover 108 to provide incident solar radiation to the top of the reaction medium 106 during daylight hours for autotrophic algal growth in the reactor 100. The reactor 100 includes a first gas sparge system 110 disposed at a higher elevation within the internal reaction volume 104 and a second gas sparge system 112 disposed at a lower elevation in the internal reaction volume 104. The reactor 100 is configured for delivery of a first pressurized sparge gas 114 to the first gas sparge system 110 and for delivery of a second pressurized sparge gas 116 to the second gas sparge system 112. The reactor 100 is also configured for continuous or periodic removal of reaction medium 106 as reactor product 118 and for supply of algal growth nutrients 120 into the internal reaction volume. During daylight hours when the reaction medium 106 is receiving incident solar radiation, a top portion of the reaction medium 106 will be in a light zone 122 within the internal reaction volume 104 adjacent the top of the reaction medium 106 and another portion of the reaction medium will be in a dark zone 124 located below in the internal reaction volume 104 the light zone 122. As used herein, light zone, or light reactor zone, refers to a zone within an internal reaction volume of an algal growth reactor occupied by algae-containing reaction medium in which the photosynthesis photon flux density (PPFD) is at or above 50 microeinsteins per square meter per second (μE m⁻² s⁻¹). The depth to which the light zone 122 shown in FIG. 1 extends below the top surface of the reaction medium 106 at any given time will depend upon particular conditions at that time in relation to incident PPFD received at the top surface of the reaction medium 106 and the composition of the reaction medium 106, such as the type and concentration of algae in the reaction medium 106. Even with very high levels of incident solar radiation where the incident PPFD may be as high as about 2,500 microeinsteins per square meter per second, the depth of the light zone 122 may be only several centimeters, for example often 8 centimeters or less, for reaction medium commonly encountered in autotrophic algal growth processes. In some preferred implementations in relation to the reactor 100, a maximum depth of the light zone 122 during algal growth processing does not extend to a depth in the internal reaction volume 104 below the first gas sparge system 110 even at times of maximum incident PPFD. As used herein, a dark zone, or dark reaction zone, refers to a zone within an internal reaction volume of an algal growth reactor occupied by reaction medium in which the PPFD is smaller than 50 microeinsteins per square meter per second. In some preferred implementations of the reactor 100 shown in FIG. 1, during autotrophic algal growth processing the top of the dark zone 122 is at a level that is at or below the first gas sparge system 110 even during times of maximum incident PPFD.

With continued reference to FIG. 1, the first gas sparge system 110 has a primary function to drive circulation of reaction medium between the light zone 122 and the dark zone 124 during autotrophic algal growth processing, with a very short residence time of reaction medium 106 in the light zone 122 so that the reactor 100 is operated in a light limitation mode with algae growth within the reactor 100 being in a linear growth regime for most or all of the time during algal growth processing. In many preferred implementations, the residence time within the light zone 122 may be on the order of milliseconds, often 5 milliseconds or less. In contrast, the residence time of reaction medium 106 in the dark zone 124 may typically be an order of magnitude or more larger than the residence time in the light zone 122. In some preferred implementations, the residence time in the dark zone 124 may be at least 0.2 second, and often even longer. By residence time of the reaction medium in a reactor zone (e.g., in the light zone 122 or the dark zone 124), it is meant the average time that reaction medium, and particularly algae within the reaction medium, spends in the reactor zone during a cycle through that reactor zone. As will be appreciated, not all portions of the reaction medium will necessarily move through a reaction zone at the exact same speed or with the same trajectory, and the residence time refers to an average time. The residence time within a reactor zone may be determined, for example, using tracer particles (e.g., radioactively labeled spheres of approximate density of reaction medium liquid) that may be tracked through an internal reaction volume as a reaction medium is being circulated within the reactor. Although the purpose of the first sparge gas 114 is primarily to drive circulation of reaction medium 106 between the light zone 122 and the dark zone 124, the first sparge gas 114 may also include some amount of carbon dioxide for use in the algal growth process. In some implementations, the first sparge gas 114 may be air, which will have a small amount of carbon dioxide useful in the algal growth reactions. The second sparge gas 116 may typically be introduced into the reaction medium 106 at a much lower velocity than the first sparge gas 114. The second sparge gas 116 may assist good circulation of the reaction medium 106 through the larger dark zone in 124 and up to the vicinity of the first gas sparge system 110 for circulation back into the light zone 122. The second sparge gas 116, however, will also typically include carbon dioxide for use in the algal growth reactions. Although the second sparge gas 116 may in some instances be air, in some preferred implementations the second sparge gas 116 may include a larger concentration of carbon dioxide than is present in air. In some preferred implementations, the second sparge gas 116 may be a gas having a high carbon dioxide content, such as may result from an anaerobic digester and/or hydrocarbon combustion. Example gas velocities for the first sparge gas 114 into the reaction medium 106 and for the second sparge gas 116 into the reaction medium 106 may for example be at a level as discussed elsewhere herein. General circulation of reaction medium in and through the light and dark zones is generally illustrated by the circulation arrows illustrating circulation by the first gas sparge system 110 and the second gas sparge system 112.

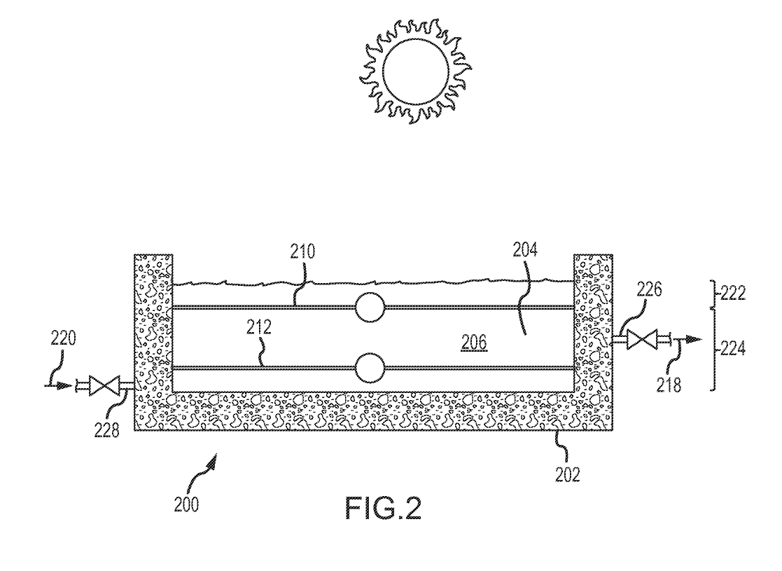

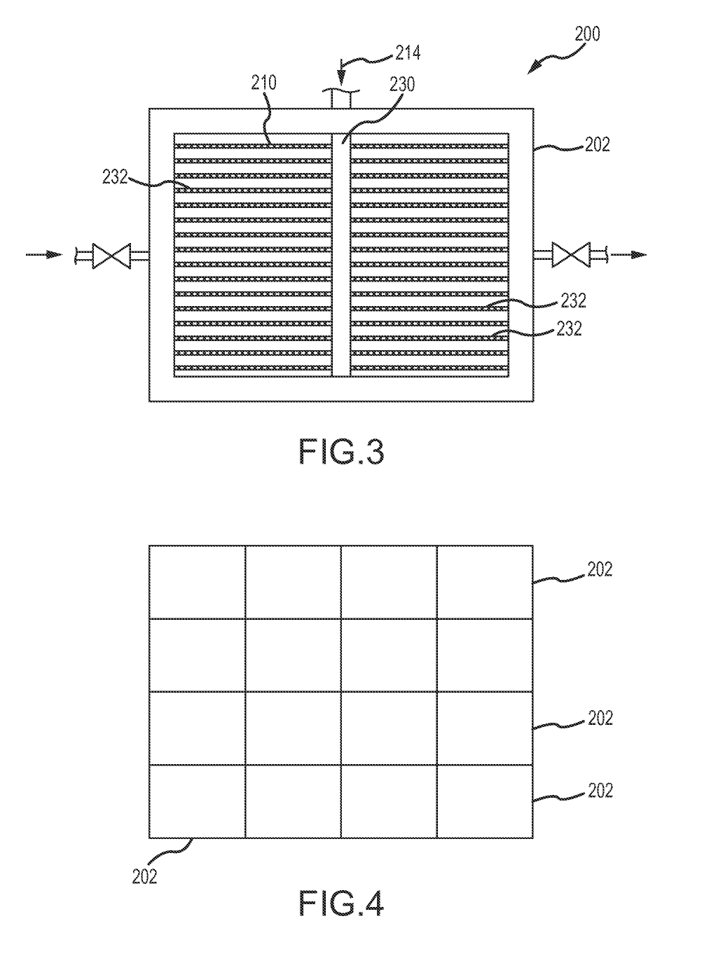

Reference is now made to FIGS. 2-5 illustrating another example embodiment of an algal growth reactor. FIGS. 2 and 3 show an example algal growth reactor 200 including a liquid-containment vessel 202 that for illustration purposes is shown in the form of a concrete-walled pond. The reactor 200 includes an internal reaction volume 204 to receive and retain reaction medium for autotrophic algal growth processing. In the illustration of FIG. 2, an example reaction medium 206 is shown disposed in the internal reaction volume 204. The reactor 200 includes a first gas sparge system 210 and a second gas sparge system 212. The first gas sparge system 210 is designed to receive and sparge into the reaction medium 206 a first sparge gas 214. The second gas sparge system 212 is disposed at a lower elevation within the internal reaction volume 204 than the first gas sparge system 210, similar to the discussion provided in relation the gas sparge systems of FIG. 1. As shown in the example illustrated in FIG. 2, the internal reaction volume 204 includes an upper light zone 222 including a top portion of the reaction medium 206 above the first gas sparge system 210 and a lower, dark zone including reaction medium 206 disposed below the first gas sparge system 210. The reactor 200 includes a reactor product removal port 226 through which reaction medium 206 may be removed as reactor product 218. The reactor 200 includes a nutrient feed port 228 through which a nutrient feed 220 may be fed into the internal reaction volume 204 for use to support algal growth in the reaction medium 206 during autotrophic algal growth processing. As illustrated in FIG. 2, the reactor 200 is shown as an uncovered pond. However, the pond could be covered to prevent rain from diluting the reaction medium 206 and to increase humidity above the top surface of the reaction medium 206 to reduce evaporative losses of liquid from the reaction medium 206. The open top of the vessel 202 provides a light transmissive path for sunlight during daylight hours to provide solar radiation to the reaction medium 206 for use in autotrophic algal growth processing. The reactor 200 may be designed in a modular manner with a specific dimensional and operational configurations, and a total reactor capacity of a desired larger size may be provided by adding reactor modules that operate in parallel. FIG. 4 illustrates an example of a large reactor capacity that is provided by a grid of 16 of the reactor vessels 202 operated independently in parallel for autotrophic algal growth processing.

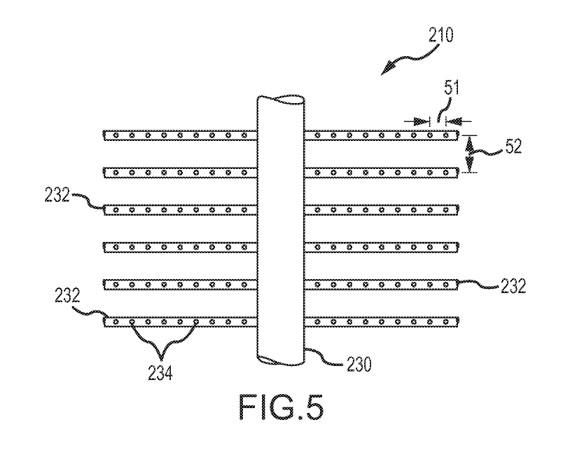

Reference is now made more specifically to FIGS. 3 and 5 to further describe aspects of the first gas sparge system 210 of the reactor 200. As shown in FIGS. 3 and 5, the first gas sparge system 210 includes a gas distribution header conduit 230 in fluid communication to feed first sparge gas 214 to a plurality of gas sparge conduits 232. Each of the sparge conduits 232 has a row of gas distribution orifices from which the first sparge gas 214 is introduced into the reaction medium 206 from the first gas sparge system 210. In some implementations, the gas distribution header 230 may be a larger-diameter pipe and the sparge conduit 232 may be smaller-diameter pipes. In the example implementation shown in FIG. 5, the gas distribution orifices 234 in a row along a sparge conduit 234 have a uniform center-to-center spacing, identified as S1 in FIG. 5. In the example shown in FIG. 5, the different rows of gas distribution orifices 234 on the different sparge conduits 232 have a uniform center-to-center spacing between the rows, identified as S2 in FIG. 5. In the example of FIG. 5, the spacing between rows of orifices (S2) is larger than the spacing between orifices in a row (S1). However, in alternative implementations, a center-to-center spacing between orifices in a row of orifices may be not uniform and/or the spacing between rows of orifices may be not uniform.

Details of the second gas sparge system 212 of the example reactor 202 are not shown. The second gas sparge system 212 may include a similar design as described with respect to the first gas sparge system 210, with orifice size, orifice spacing and a density of orifices for gas flows to be provided in the second gas sparge system 212. In that regard, gas velocities from gas distribution orifices in the first gas sparge system 210 will be typically significantly larger than gas velocities from gas distribution orifices of the second gas sparge system 212.

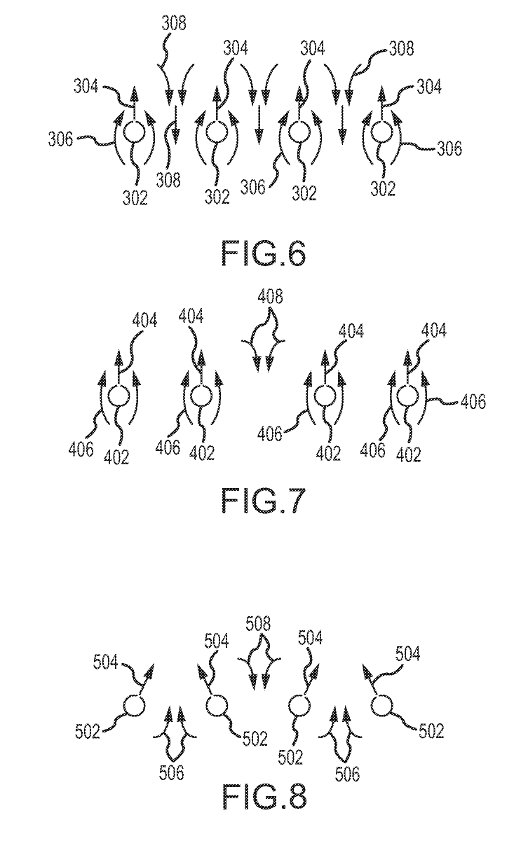

Reference is now made to FIGS. 6-8 to illustrate some example configurations for sparge gas distribution in a gas sparge system to drive reaction medium circulation between light and dark reactor zones, for example in the first gas sparge system 110 of FIG. 1 or the second gas sparge system 210 of FIGS. 2-5. Referring first to FIG. 6, a plurality of example gas sparge conduits 302 are shown in cross section illustrating gas flow from an example gas distribution orifice of a row of orifices that may be disposed along each gas sparge conduit 302. Gas flow from each orifice is directed vertically upward from the orifices as generally illustrated by the sparge gas flow arrows 304. The upward sparge gas flow creates a low pressure area that pulls flow of reaction medium from below to above the gas sparge conduits 302, for example from a lower dark reactor zone, upward into a light reactor zone. Such upward flow of reaction medium is generally illustrated by the upward flow arrows 306. Circulation of reaction medium back to the dark reactor zone below the gas sparge conduits 302 may be provided by reaction medium falling through the middle portion of the space between rows of the gas sparge conduits 302, illustrated generally by the downward flow arrows 308.

Referring now to FIG. 7, another example configuration is shown for gas distribution orifices for a gas sparge system to drive reaction medium circulation between a light reactor zone and a dark reactor zone, for example the first gas sparge system 110 of FIG. 1 or the second gas sparge system 210 of FIGS. 2-5. FIG. 7 illustrates a plurality of gas sparge conduits 402 each with a row of gas distribution orifices configured for introducing sparge gas flow vertically upward into the reaction medium similar to gas flow in FIG. 6 and generally illustrated in FIG. 7 by the upward flow arrows 404. In the configuration shown in FIG. 7, the gas sparge conduits 402 are arranged in pairs with a closer spacing between gas sparge conduits 402 in a pair and a larger spacing between such pairs of gas sparge conduits 402. The larger spacing between pairs of the gas sparge conduits 402 may provide a larger flow path to provide a preferential return path for downward flow of reaction medium to cycle back to a dark zone below the gas sparge conduits 402. Such downward flow of reaction medium is generally illustrated by the downward flow arrows 408. Some downward flow of reaction medium may also occur between gas sparge conduits 402 in a pair.

Reference is now made to FIG. 8 illustrating another example configuration for gas distribution orifices for a gas sparge system to drive circulation of reaction medium between a light reactor zone and a dark reactor zone, for example in the first gas sparge system 110 of FIG. 1 or the first gas sparge system 210 of FIGS. 2-5. FIG. 8 shows a plurality of evenly spaced gas sparge conduits 502. However, in contrast to the configurations shown in FIGS. 6 and 7, the gas distribution orifices in the gas sparge conduits 502 of FIG. 8 are oriented to provide upward sparge gas flow at a slight angle to vertical so that gas flow from a pair of adjacent ones of the gas sparge conduits 502 will tend to converge at an elevation above the sparge gas conduits 502. Such a gas distribution configuration may provide alternating preferential flow paths for upward and downward flow of reaction medium for circulation of the reaction medium between light and dark reactor zones. Such preferential paths for upward flow of reaction medium are shown generally by the upward flow arrows 506 and such preferential paths for downward flow paths for reaction medium are shown generally by the downward flow arrows 508.

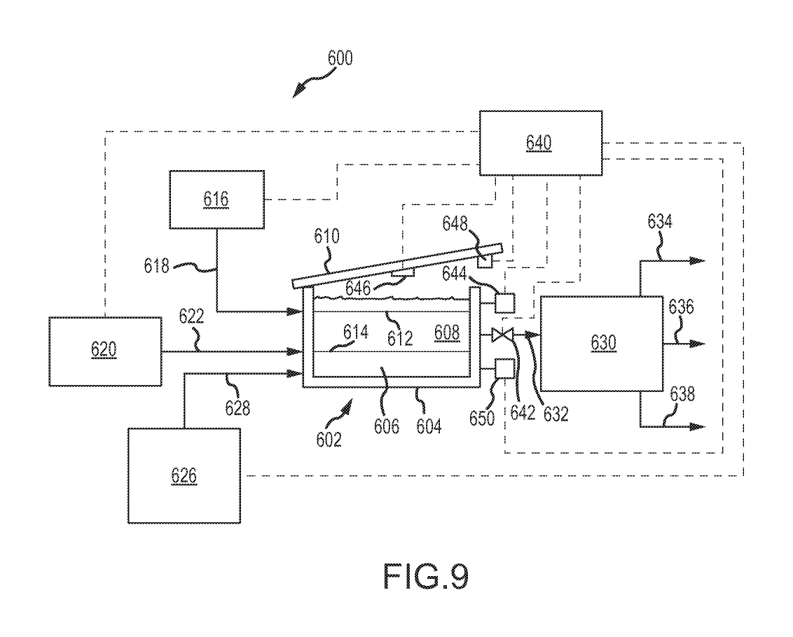

Reference is now made to FIG. 9, which illustrates an example algal growth system 600 for autotrophic algal growth. The algal growth system 600 includes an algal growth reactor 602 including a liquid-containment vessel 604 with an internal reaction volume 606 to receive and contain algae-containing reaction medium 608 during autotrophic algal growth processing. The reactor 602 includes a cover 610 that prevents rainwater from diluting the reaction medium 608 inside the vessel 604 and to provide increased humidity above the top of the reaction medium 608 to reduce evaporative losses of aqueous liquid from the reaction medium 608. The reactor 602 includes a first gas sparge system 612 disposed at a higher elevation within the internal reaction volume 606 and a second gas sparge system 614 disposed at a lower elevation within the internal reaction volume 606. The first gas sparge system 612 may provide a primary mechanism for driving circulation of reaction medium 608 between a light reactor zone above the first gas sparge system 612 and a dark reactor zone below the first gas sparge system 612. The second gas sparge system 614 may assist circulation within the internal reaction volume and may provide a source for additional carbon dioxide for algal growth. The cover 610 is optically transmissive and together with the open area below the cover 610 to the top of the reaction medium 608 provides an optically transmissive path for providing solar radiation to the reaction medium in the light reactor zone for autotrophic algal growth during daylight hours.

The algal growth system 600 includes a first sparge gas delivery system 616 in fluid communication with the first gas sparge system 612 to provide a feed of pressurized first sparge gas 618 to the first gas sparge system 612 as needed for autotrophic algal growth processing. A second sparge gas delivery system 620 is in fluid communication with the second gas sparge system 614 to provide feed of a pressurized second sparge gas 622 to the second gas sparge system 614 as needed during autotrophic algal growth processing. The first sparge gas delivery system 620 may include a source for compressed first sparge gas, for example compressed air. The first sparge gas delivery system may include, for example, one or more air compressors, pressure accumulators, valves and/or pressure regulators. The second sparge gas delivery system 620 may include a source for compressed second sparge gas, for example as may be sourced from an anaerobic digester and/or from combustion exhaust gas. The second gas delivery system may include, for example, one or more gas compressors, pressure accumulators, valves and/or pressure regulators. In some alternative implementations, the second gas sparge system 620 may supply compressed air as the second sparge gas 622, in which case the first gas sparge system 616 and the second gas sparge system 620 may be combined to an extent combination is convenient.

The algal growth system 600 includes a nutrient supply system 626 in fluid communication with the internal reaction volume 606 to supply nutrient feed 628 to the internal reaction volume 606 as needed for autotrophic algal growth processing. The nutrient feed 628 may be provided as a single feed stream or as multiple feeds streams. A feed stream may include a liquid with one or more nutrients dissolved and/or dispersed therein. Such nutrients may include, for example, one or more than one member selected from the group consisting of nitrogen nutrients, phosphorous nutrients, sodium nutrients, potassium nutrients, magnesium nutrients, calcium nutrients, vitamins, iron and trace metal. The nutrient supply system may include, for example, one or more vessels containing a supply of the nutrient feed 628 or components of or precursors for the nutrient feed 628 and associated equipment such as pumps and/or valves.

The algal growth system 600 also includes a product recovery system 630 in fluid communication with the internal reaction volume 606 to receive portions of the reaction medium 608 that may be withdrawn from the internal reaction volume 606 as reactor product 632 containing a desired concentration of algae. In the product recovery system, algae recovered as the reactor product 632 may be lysed, before or after dewatering, and the resulting lysed material may be separated into a lipid fraction 634, an aqueous fraction 636 and a solids fraction 638. The lipid fraction 634 may be advantageously recovered for use as or for further processing to prepare a biofuel product. The aqueous liquid fraction 636 may be recycled, with appropriate treatment as necessary, for further use within the algal growth system 600. The solids fraction 638, including residual biomass material, may be recovered as a fertilizer product to be sold or may be subjected to anaerobic digestion, for example to prepare methane and carbon dioxide. Such methane may be used to generate electricity and carbon dioxide, including that generated by combustion of the methane, may be recycled within the algal growth system 600, for example for use as or to prepare the second sparge gas 622 in the second sparge gas delivery system 620. The product recovery system may include, for example, appropriate equipment such as process vessels, separators, pumps and/or valves.

The algal growth system 600 includes a computer controller system 640 to control various reactor operating parameters to control autotrophic algal growth in the internal reaction volume 606. The computer controller system 640 is in communication, for example in electronic or optical signal communication, with the first gas sparge delivery system 616, the second gas sparge delivery system 620, the nutrient supply system 626 and a product control valve 642 on a conduit for the reactor product 632. The computer controller may generate control signals, for example electronic or optical control signals, to adjust one or more reactor operating parameters. For example, control signals may be directed to the first sparge gas delivery system 616 to control the supply of the first sparge gas feed 618 to the first gas sparge system 612, for example to turn the flow of the first sparge gas feed 618 on and off or to control the pressure at which the first sparge gas feed 618 is provided to the first gas sparge system 612. As another example, the computer controller system 640 could provide control signals to the second sparge gas delivery system 620 to control supply of the second sparge gas feed 622 to the second gas sparge system 614, for example in a similar manner as control may be directed to the first sparge gas delivery system 616. The computer controller system 640 may provide control signals to the nutrient supply system 626 to control supply of the nutrient feed 628 to the internal reactor volume 606. Such control may include turning on and off the nutrient feed 628 as needed, adjusting a rate at which the nutrient feed 628 is supplied to the internal reaction volume 606 and/or changing the composition of the nutrient feed 628 (e.g., to change relative amounts of different nutrient components). The computer controller system 640 may provide control signals to the product control valve 642 to control withdrawal of reaction medium 608 as reactor product 632 for recovery and processing in a product recovery system 630. The control of the product control valve 642 may include, for example, to open and close the control valve 642 or to adjust the valve to adjust a rate at which reactor product 632 is recovered from the reactor 602.

The algal growth system 600 also includes a monitoring system to monitor various properties during autotrophic algal growth in the internal reaction volume and to generate and transmit data signals (for example, electronic data signals or optical data signals) with data indicative of monitored properties. Such data signals may be received and processed by the computer controller system 640 to generate appropriate control signals. In the example algal growth system 600 shown in FIG. 9, the monitoring system includes a pulse-amplitude modulated fluorometer unit 644, a passive fluorometer unit 646, an incident light monitoring unit 648 and a dissolved nitrogen monitoring unit 650. The pulse-amplitude modulated fluorometer unit 644 may periodically sample reaction medium 608 in the internal reaction volume 606 and subject the sample to pulse-amplitude modulated fluorometry, and based on the monitored property transmit data signals indicative of monitored pulse-amplitude modulated fluorometry results to the computer controller system 640. The passive fluorometer unit 646 may monitor fluorescent emissions from the reaction medium 608 in the light zone of the reactor 602 due to excitation by solar radiation incident upon the reaction medium during autotrophic algal growth processing. The passive fluorometer unit 646 may generate and transmit to the computer controller system 640 data signals indicative of monitored fluorescent emissions. The incident light monitoring unit 648 may include a light sensor for sensing a range of wave lengths of photosynthetically active radiation to monitor a level of incident PPFD being received by the reaction medium 608 and to generate and transmit to the computer controller system 640 data signals indicative of monitored light. The dissolved nitrogen monitoring unit 650 may monitor a concentration of dissolved nitrogen in liquid of the reaction medium 608 and may generate and transmit to the computer controller system 640 data signals indicative of monitored nitrogen concentrations. As used herein, dissolved nitrogen and dissolved nitrogen concentration refer to all nitrogen contained in nitrogen-containing solutes in aqueous liquid of the reaction medium 608, regardless of the particular chemical constituent group in which the nitrogen is present (e.g., ammonium group, nitrate group or other group). The computer controller system 640 may include a computer processor and non-volatile computer memory with instructions executable by the computer processor to evaluate electronic data signals received by the computer controller system 640 and to generate electronic control signals.

During operation of the algal growth system 600, feed streams to the reactor 602 and recovery of reactor products 632 may be turned off during hours of insufficient solar radiation for desired autotrophic algal growth processing, for example during nighttime hours, and may be turned on as needed for autotrophic algal growth processing when sufficient incident solar radiation is received by the reaction medium 608 during daylight hours, for example as sensed by the incident light monitoring unit 648 and controlled by the computer controller system 640. During algal growth processing, incident PPFD may be monitored by the incident light monitor 648 and the computer controller system 640 may control operating parameters to adjust the residence time of reaction medium 608 within the light zone in the internal reaction volume 606 to maintain the reaction medium 608 in a linear growth regime where the rate of algal biomass production is proportional to incident PPFD. Such control may include, for example adjusting feed pressure of the first sparge gas feed 618 and/or adjusting the level of the reaction medium 608 above the first gas sparge system 612. Likewise, fluorometric monitoring provided by the pulse-amplitude modulated fluorometer unit 644 and/or the passive fluorometer unit 646 may indicate that incident PPFD is not being used efficiently for algal growth and the computer controller system 640 may make similar adjustments to adjust the residence time of reaction medium 608 in the light zone of the internal reaction volume 606, for example by adjusting feed pressure of the first sparge gas 618 and/or the level of the reaction medium 608 above the first gas sparge system 612. Changing a level of the reaction medium 608 above the first gas sparge system 612 may include, for example increasing or decreasing a rate of reaction medium 608 removed from the internal reaction volume 606 as reactor product 632 and/or a rate of addition of nutrient feed 628 to the internal reaction volume 606. Moreover, the computer controller system 640 may adjust a rate of nutrient feed 628 to the internal reaction volume 606 for algal growth requirements based on incident PPFD level received by the reactor 602 and/or a level of monitored dissolved nitrogen concentration.

The foregoing discussion of the invention and different aspects thereof has been presented for purposes of illustration and description. The foregoing is not intended to limit the invention to only the form or forms specifically disclosed herein. Consequently, variations and modifications commensurate with the above teachings, and the skill or knowledge of the relevant art, are within the scope of the present invention. The embodiments described hereinabove are further intended to explain best modes known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art. Although the description of the invention has included description of one or more possible implementations and certain variations and modifications, other variations and modifications are within the scope of the invention, e.g., as may be within the skill and knowledge of those in the art after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. Furthermore, any feature described or claimed with respect to any disclosed implementation may be combined in any combination with one or more of any other features of any other implementation or implementations, to the extent that the features are not necessarily technically compatible, and all such combinations are within the scope of the present disclosure.

The terms “comprising”, “containing”, “including” and “having”, and grammatical variations of those terms, are intended to be inclusive and nonlimiting in that the use of such terms indicates the presence of some condition or feature, but not to the exclusion of the presence also of any other condition or feature. The use of the terms “comprising”, “containing”, “including” and “having”, and grammatical variations of those terms in referring to the presence of one or more components, subcomponents or materials, also include and is intended to disclose the more specific embodiments in which the term “comprising”, “containing”, “including” or “having” (or the variation of such term) as the case may be, is replaced by any of the narrower terms “consisting essentially of” or “consisting of” or “consisting of only” (or the appropriate grammatical variation of such narrower terms). For example, a statement that some thing “comprises” a stated element or elements is also intended to include and disclose the more specific narrower embodiments of the thing “consisting essentially of” the stated element or elements, and the thing “consisting of” the stated element or elements. Examples of various features have been provided for purposes of illustration, and the terms “example”, “for example” and the like indicate illustrative examples that are not limiting and are not to be construed or interpreted as limiting a feature or features to any particular example. The term “at least” followed by a number (e.g., “at least one”) means that number or more than that number. The term at “at least a portion” means all or a portion that is less than all. The term “at least a part” means all or a part that is less than all. Operations or steps of any method or process need not be performed in any particular order unless a particular order is required.

1-48. (canceled)

49. A method for autotrophic algal growth, the method comprising:
circulating an algae-containing reaction medium between a light reactor zone and a dark reactor zone of an internal reaction volume of an algal growth reactor;
during the circulating, adding to the reaction medium nutrients for algal growth in the reaction medium, the nutrient comprising at least a nitrogen nutrient;
during the circulating, irradiating the reaction medium in the light reactor zone with photosynthetically active radiation for absorption by algae in the algae-containing medium for algal photosynthesis; and
during the circulating, maintaining a first residence time of the reaction medium in the dark reactor zone of at least 0.2 second and a second residence time of the reaction medium in the light reactor zone of not more than 5 milliseconds;
and wherein:
a ratio of the first residence time to the second residence time is at least 100:1;
the circulating comprises sparging gas into the reaction medium at a gas velocity of at least 2 meters per second;
the sparging comprises introducing the gas into the reaction medium from gas delivery ports having a maximum cross-dimension perpendicular to a direction of flow in a range of from 2 microns to 200 microns; and
during the circulating, the dark reactor zone contains a first volume of the reaction medium and the light reactor zone contains a second volume of the reaction medium, wherein a ratio of the first volume to the second volume is at least 5:1.

50. A method according to claim 49, wherein:
the algal growth reactor comprises a reactor vessel in which the light reactor zone is disposed at a higher elevation within the reactor vessel than the dark reactor zone; and
the irradiating comprises receiving natural sunlight into the reactor vessel from above.

51. A method according to claim 50, wherein the reactor vessel is open to the exterior environment.

52. A method according to claim 50, comprising:
during the circulating, removing a portion of the reaction medium from the reactor as reactor product; and
monitoring a nitrogen solution concentration of nitrogen in liquid of the reactor product and adjusting an amount of the nitrogen nutrient added to the reaction medium during the adding to maintain the nitrogen solution concentration in the reactor product within a range of from 14 micrograms to 700 micrograms of dissolved nitrogen per liter of the liquid.

53. A method according to claim 52, wherein during the circulating, the reactor is operated at a nitrogen quotient in a range of from 50% to 95% of the nitrogen quotient measured in the same algal culture under nitrogen excess, wherein the nitrogen quotient is in grams of nitrogen in biomass of the reactor product per gram of the biomass on a dry weight basis.

54. A method according to claim 53, wherein at least 90 weight percent of biomass, on a dry weight basis, in the reactor product is eukaryotic algae.

55. A method according to claim 49, wherein:
the reaction medium in the light reactor zone has a quiescent depth of not larger than 8 centimeters; and
the dark reactor zone has a depth from top to bottom in a range of from 20 centimeters to 100 centimeters.

56. A method according to claim 49, comprising monitoring the incident photosynthesis photon flux density (PPFD) of the electromagnetic radiation onto the algal culture and adjusting at least one operating parameter of the reactor based on changes in the monitored incident PPFD, wherein the at least one operating parameter includes a member selected from the group consisting of residence time of the reaction medium in the light reactor zone, rate of addition of nitrogen, depth of liquid in the light reactor zone and considerations thereof.

57-58. (canceled)

59. A method for autotrophic algal growth, the method comprising:
circulating an algae-containing reaction medium between a light reactor zone and a dark reactor zone of an internal reaction volume of an algal growth reactor;
during the circulating, adding to the reaction medium nutrients for algal growth in the reaction medium, the nutrient comprising at least a nitrogen nutrient;
during the circulating, irradiating the reaction medium in the light reactor zone with photosynthetically active radiation for absorption by algae in the algae-containing medium for algal photosynthesis;
during the circulating, maintaining a first residence time of the reaction medium in the dark reactor zone of at least 0.2 second and a second residence time of the reaction medium in the light reactor zone of not more than 5 milliseconds; and
during the irradiating, fluorometrically monitoring the reaction medium and adjusting at least one operating parameter of the reactor in response to a change in a monitored fluorometric property of the reaction medium, wherein the adjusting comprises decreasing residence time of reaction medium in the light reactor zone in response to an increase in monitored fluorescence of the reaction medium during the fluorometric monitoring.

60-62. (canceled)

63. An algal growth system for autotrophic algal growth, comprising:
an algal growth reactor with an internal reaction volume to receive and contain algae-containing reaction medium during autotrophic algal growth;
the reactor comprising a first reactor portion including a first portion of the internal reaction volume to provide a dark reactor zone for the reaction medium during autotrophic algal growth;
the reactor comprising a second reactor portion including a second portion of the internal reaction volume to provide a light reactor zone for the reaction medium during autotrophic algal growth;
a light transmissive path in optical communication with the second portion of the internal reaction volume to provide photosynthetically active radiation from a light source to the light reactor zone of the second portion of the internal reaction volume to be absorbed by biomass in the second portion of the internal reaction volume during autotrophic algal growth;
a ratio of the volume of the first portion of the internal reaction volume to the volume of the second portion of the internal reaction volume of at least 5:1; and
a liquid circulation system to circulate the reaction medium during autotrophic algal growth between the dark reactor zone in the first portion of the internal reaction volume and the light reactor zone in the second portion of the internal reaction volume, the liquid circulation system comprising a gas sparge system to sparge pressurized gas into the internal reaction volume between the first portion and the second portion of the internal reaction volume to drive circulation of the reaction medium between the dark reactor zone in the first portion of the internal reaction volume and the light reactor zone in the second portion of the internal reaction volume during autotrophic algal growth;
and wherein:
the gas sparge system comprises a plurality of gas delivery ports to deliver compressed gas into the internal reaction volume between the first portion and the second portion of the internal reaction volume;
the gas delivery ports have a maximum cross-dimension perpendicular to a direction of flow of gas from the gas delivery ports in a range from 2 microns to 200 microns; and
the gas sparge system includes an array of the gas delivery ports at a density of the gas delivery ports of from 200 to 20,000 of the ports per square meter.

64. An algal growth reactor according to claim 63, wherein the gas delivery ports are in spaced rows of orifices with a first center-to-center spacing between orifices in a row being smaller then a second center-to-center spacing between said rows.

65. An algal growth reactor according to claim 64, wherein the second center-to-center spacing is at least 1.5 times as large as the first center-to-center spacing.

66-89. (canceled)
